(19) United States
(12) Patent Application Publication
(10) Pub. No.: US 2018/0222848 A1
(43) Pub. Date: Aug. 9, 2018

(54) Salt of Dihydrophenylglycine Methyl Ester

(71) Applicant: DSM Sinochem Pharmaceuticals Netherlands B.V., Delft (NL)

(72) Inventors: Thomas VAN DER DOES, Delft (NL); Harold Monro MOODY, Delft (NL); Weidong LI, Delft (NL)

(21) Appl. No.: 15/750,195

(22) PCT Filed: Aug. 2, 2016
(86) PCT No.: PCT/EP2016/068384

§ 371 Date: Feb. 4, 2018

(30) Foreign Application Priority Data
Aug. 4, 2015 (EP) 15179710.7

(51) Int. Cl. C07C 229/32; C12P 37/00

(52) U.S. Cl. CPC C07C 229/32; C07C 2601/16; C12Y 305/01011; C12P 37/00

(57) ABSTRACT

The present invention relates to the hemi sulfuric acid salt of methyl (R)-2-amino-2-(cyclohexa-1,4-dien-1-yl)acetate, also trivially referred to as the hemi sulfuric acid salt of D-dihydrophenylglycine methyl ester, to a method for the preparation of said salt and to the use of said salt in the enzymatic synthesis of antibiotics.

### FIELD OF THE INVENTION

The present invention relates to the hemi sulfuric acid salt of methyl (R)-2-amino-2-(cyclohexa-1,4-dien-1-yl)acetate, also trivially referred to as the hemi sulfuric acid salt of D-dihydrophenylglycine methyl ester, to a method for the preparation of said salt and to the use of said salt in the enzymatic synthesis of antibiotics.

### BACKGROUND OF THE INVENTION

Enzymatic production of semisynthetic β-lactam antibiotics by acylation of the parent amino β-lactam moiety with a side chain acid derivative, such as an amide or an ester, has been widely described in the patent literature e.g. DE 2163792, DE 2621618, EP 339751, EP 473008, U.S. Pat. No. 3,816,253, WO 92/01061, WO 93/12250, WO 96/02663, WO 96/05318, WO 96/23796, WO 97/04086, WO 98/56946, WO 99/20786, WO 2005/003367, WO 2006/069984, WO 2008/110527 and WO 2011/073166. The enzymes used in the art are in most cases penicillin acylases obtained from Escherichia coli and are immobilized on various types of water-insoluble materials (e.g. WO 97/04086).

Due to the sensitive nature of biocatalysts, enzymatic processes usually have strict requirements with regard to the presence of contaminants. Often, unwanted impurities disturb the proper functioning of an enzyme. For this reason, also in the enzymatic production of semisynthetic β-lactam antibiotics by acylation of the parent amino β-lactam moiety with a side chain acid derivative, such as an amide or an ester, the starting materials are preferably in the highest possible purity. The latter is usually achieved by isolating the starting materials, preferably by means of crystallization. For example, for D-4-hydroxyphenylglycine, the side chain for antibiotics such as amoxicillin, cefadroxil and cefprozil, crystallization of activated derivatives such as amides or esters can be easily achieved. For (R)-2-amino-2-(cyclohexa-1,4-dien-1-yl)acetic acid (trivially referred to as D-dihydrophenylglycine, DHPG), the side chain for antibiotics such as cefroxadine, cephradine and epicillin, this is however a major problem. Up to now there have not been any reports on the isolation of crystalline methyl (R)-2-amino-2-(cyclohexa-1,4-dien-1-yl)acctate, one of the most favored starting materials in enzymatic production of epicillin and cephradine. As described in WO 2008/110527, there is however a need for highly purified methyl (R)-2-amino-2-(cyclohexa-1,4-dien-1-yl)acetate, as the presence of traces of (R)-2-amino-2-(cyclohexa-1,4-dien-1-yl)acetic acid has a strong negative effect on the yield of the enzymatic coupling reaction. This is attributed to the fact that, due to the low solubility of the free side chains under the conditions of the enzymatic coupling reaction, there is an upper limit to the concentration of free side chain in the enzymatic coupling reaction. This limit is determined by the requirement that the free side chain should not crystallize or precipitate, because the precipitate negatively affects the processing of the enzymatic coupling reaction. Moreover, in the final steps of the downstream processing of the semi synthetic β-lactam compound, the contaminating (R)-2-amino-2-(cyclohexa-1,4-dien-1-yl)acetic acid has to be removed, for instance with the mother liquor of a final crystallization step of the semi synthetic β-lactam compound. At higher levels of (R)-2-amino-2-(cyclohexa-1,4-dien-1-yl)acetic acid, more mother liquor is required to remove the (R)-2-amino-2-(cyclohexa-1,4-dien-1-yl)acetic acid which in turn is responsible for higher losses of the semi synthetic β-lactam compound. The unit operation which results in the isolation of the side chain ester in solid form complicates the production process of the semi synthetic antibiotic and significantly contributes to the cost price thereof. Therefore the amount of unwanted (R)-2-amino-2-(cyclohexa-1,4-dien-1-yl)acetic acid in methyl (R)-2-amino-2-(cyclohexa-1,4-dien-1-yl)acetate should be as low as possible.

In order to achieve this, methyl (R)-2-amino-2-(cyclohexa-1,4-dien-1-yl)acetate can be isolated in the form of a salt. Several salts such as alkyl- or aryl sulfonic acid salts and the hydrochloric acid have been reported and through such isolation process unwanted traces of D-phenylglycine can be removed. However, these salts bring certain disadvantages such as the introduction of new organic impurities. Also, said salts are often obtainable in solid form only by precipitation techniques which usually bring insufficient purification as similar components, in this case the contaminating (R)-2-amino-2-(cyclohexa-1,4-dien-1-yl)acetic acid, co-precipitate. For example, WO 2007/039522 describes sulfonic acid salts of methyl (R)-2-amino-2-(cyclohexa-1,4-dien-1-yl)acetate. However, although the product as described in this document can be used for the enzymatic synthesis of cephradine, there still is a need for highly pure salts of methyl (R)-2-amino-2-(cyclohexa-1,4-dien-1-yl)acetate in order to further improve yield and efficiency of enzymatic synthesis of antibiotics such as cefroxadine, cephradine and epicillin. In principle the hydrochloric acid salt is an attractive candidate for isolation of a purified derivative of methyl (R)-2-amino-2-(cyclohexa-1,4-dien-1-yl)acetate but unfortunately, the penicillin acylases are a class of enzymes that is negatively influenced by the presence of chloride salts and therefore the use of the hydrochloric acid salt of methyl (R)-2-amino-2-(cyclohexa-1,4-dien-1-yl)acetate in enzymatic synthesis is accompanied with additional problems that are of a larger magnitude than the problem originally set out to solve. It is for this reason that there remains a need for derivatives of methyl (R)-2-amino-2-(cyclohexa-1,4-dien-1-yl)acetate that can be isolated, are of sufficient purity and do not have the problem associated with the hydrochloric acid salt of methyl (R)-2-amino-2-(cyclohexa-1,4-dien-1-yl)acetate, such as for example reactor corrosion by chloride ions.

### DETAILED DESCRIPTION OF THE INVENTION

It is an object of the present invention to provide a derivative of methyl (R)-2-amino-2-(cyclohexa-1,4-dien-1-yl)acetate that can be isolated, is of sufficient purity and can be used without inhibiting side effects in enzymatic processes leading to cefroxadine, cephradine and epicillin.

The term “nucleus”,is defined herein as the β-lactam moiety of the semi synthetic β-lactam and may be any penem or cephem, for instance 7-aminocephalosporanic acid (7-ACA) or 7-amino-3-chloro-3-cephem-4-carboxylic acid (7-ACCA) or 7-amino-deacetoxycephalosporanic acid (7-ADCA) or 7-amino-3-methoxy-3-cephem-4-carboxylic acid (7-AMOCA) or 6-aminopenicillanic acid (6-APA), preferably 6-APA, 7-ADCA or 7-AMOCA as these nuclei are the precursors to pharmaceutically relevant semi synthetic β-lactams epicillin, cephradine and cefroxadine, respectively.

The term “side chain”,is defined herein as the moiety which in the semi synthetic β-lactam compound is attached to the 6-amino or 7-amino position in the nucleus as defined herein, i.e. (R)-2-amino-2-(cyclohexa-1,4-dien-1-yl)acetyl in cefroxadine, cephradine and epicillin.

The term “free side chain”,is the un-derivatized form of the side chain, i.e. (R)-2-amino-2-(cyclohexa-1,4-dien-1-yl)acetic acid (also trivially referred to as D-dihydrophenylglycine).

The term “side chain ester”,is the ester form of the free side chain whereby the carboxyl group of the free side chain is esterified to an alcohol, for instance methyl (R)-2-amino-2-(cyclohexa-1,4-dien-1-yl)acetate (also trivially referred to as D-dihydrophenylglycine methyl ester). The side chain ester may be in the form of the free base or as a salt, for instance as the sulfuric acid salt.

The term “hemi sulfuric acid salt of methyl (R)-2-amino-2-(cyclohexa-1,4-dien-1-yl)acetate”, abbreviated as (DHPGMH)₂SO₄, refers to the compound of formula (1), with formula C₁₈H₂₈N₂SO₈.

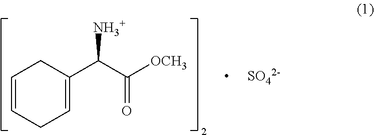

In the art the compound of formula (1) is also trivially referred to as hemi sulfuric acid salt of D-dihydrophenylglycine methyl ester, although several different isomers is having the same molecular formula also exist. In the context of the present invention “hemi sulfuric acid salt of D-dihydrophenylglycine methyl ester”,refers only to the compound of formula (1).

In a first aspect, the invention provides the hemi sulfuric acid salt of methyl (R)-2-amino-2-(cyclohexa-1,4-dien-1-yl)acetate, (DHPGMH)₂SO₄, in isolated form. Preferably said (DHPGMH)₂SO₄ is crystalline. The formation of crystalline (DHPGMH)₂SO₄ is surprising since reports of crystalline forms of salts of these amino acid methyl esters have hitherto not been reported. WO 2007/039522, for example, does disclose isolated sulfonic acid salts, however these are obtained by precipitation, not crystallization and therefore of inferior purity. Clearly this aspect brings unexpected advantages in terms of end-product purity and (lack of) enzyme inhibition which indeed is observed in use tests. In one embodiment crystalline (DHPGMH)₂SO₄ has an XRD powder diffraction pattern as given in FIG. 1. Preferably said XRD powder diffraction pattern reveals peaks at 5.9±0.2 degrees 2-theta, 11.8±0.2 degrees 2-theta, 19.2±0.2 degrees 2-theta and 23.8±0.2 degrees 2-theta. More preferably said XRD powder diffraction pattern reveals additional peaks at 16.4±0.2 degrees 2-theta, 22.0±0.2 degrees 2-theta and 25.3±0.2 degrees 2-theta.

The (DHPGMH)₂SO₄ of the present invention advantageously is a stable solid. The only other known stable inorganic acid salt of methyl (R)-2-amino-2-(cyclohexa-1,4-dien-1-yl)acetate is the hydrochloric acid salt. However the latter salt has some drawbacks such as a negative influence on enzyme performance and release of corrosive chloride as side product. The formation of chlorides is known to have a detrimental effect on industrial reactors and this phenomenon does not occur with the sulfates that are being formed with the use of the (DHPGMH)₂SO₄ of the present invention. Surprisingly, application of the (DHPGMH)₂SO₄ of the present invention in the enzymatic synthesis of semi synthetic (R)-2-amino-2-(cyclohexa-1,4-dien-1-yl)acetyl-comprising β-lactam compounds such as cefroxadine, cephradine or epicillin resulted in improved results such as shorter reaction times and reduced use of biocatalyst when compared to the use of a pre-formed solution of methyl (R)-2-amino-2-(cyclohexa-1,4-dien-1-yl)acetate as advocated in WO 97/04086 and WO 2011/073166. In one embodiment, the antibiotic cephradine can be prepared enzymatically from 7-ADCA in shorter reaction times, with higher conversion and lower formation of unwanted cephalexin using the (DHPGMH)2504 of the present invention.

In another embodiment, the (DHPGMH)₂SO₄ of the present invention is dissolved in water such as to provide an aqueous solution. In certain applications, such a solution greatly facilitates accurate addition protocols in enzymatic reactions compared to solid crystalline (DHPGMH)₂SO₄.

In a second aspect, the invention provides a method for the preparation of (DHPGMH)₂SO₄ comprising the steps of:
(a) contacting methyl (R)-2-amino-2-(cyclohexa-1,4-dien-1-yl)acetate free base with sulfuric acid;
(b) isolating the hemi sulfuric acid salt of methyl (R)-2-amino-2-(cyclohexa-1,4-dien-1-yl)acetate from the mixture obtained in step (a).

Preparation of methyl (R)-2-amino-2-(cyclohexa-1,4-dien-1-yl)acetate free base may be carried out using methods known to the skilled person, such as for example described in WO 2008/110527.

Isolating may be carried out by separating the formed crystalline product from the mixture obtained in step (a) by techniques known to the skilled person such as centrifugation, filtration and sedimentation/decantation and the like.

Preferably, the amount of sulfuric acid is chosen such that the molar amount of sulfuric acid is from 0.4 to 0.6 relative to the molar amount of (DHPGMH)₂SO₄. When step (a) is carried out in aqueous environment, in a preferred embodiment, (DHPGMH)₂SO₄ is isolated by separating the aqueous phase in step (a) and crystallizing (DHPGMH)₂SO₄ therefrom.

Crystallization may be carried out or promoted according to methods known to the skilled person, for example by lowering the temperature. It was found that a preferred crystallization temperature is from −5 to 15° C., more preferably from 0 to 10° C.

In one embodiment, it was found that the overall yield can be improved by recycling the mother liquor remaining after the isolation in step (b) of the above method. Thus, the mother liquor is added to the mixture of step (a) in a next cycle of the method as described above. Preferably recycling is carried out such that part of the mother liquor is discarded prior to addition to the mixture of step (a). A suitable small part is from 1- to 50% by volume, preferably from 2 to 25% by volume, more preferably from 3 to 15% by volume. As a result of the phase separation it was found that this recycling can be performed without accumulation of impurities.

The method of the second aspect can also be carried out with various organic solvents. It was found that preferred solvents are those having a solubility in water of from 0% (w/w) to 25% (w/w) and having a polarity index of from 1 to 5. Preferably said polarity index is from 2 to 3 as this generally leads to the best results. Preferred solvents are butyl acetate, diethyl ether, ethyl acetate, methyl isobutyl ketone and methyl tert-butyl ether.

Advantageously, the method of the second aspect is superior to the obvious alternative wherein DHPG is reacted with methanol in the presence of sulfuric acid. In our hands the latter method failed due to esterification of sulfuric acid to hydrogen methylsulfate.

In a third aspect, the invention provides the use of (DHPGMH)₂SO₄ in the preparation of cefroxadine, cephradine or epicillin comprising contacting said (DHPGMH)₂SO₄ with 7-amino-3-methoxy-3-cephem-4-carboxylate (7-AMOCA), 7-aminodeacetoxycephalosporanic acid (7-ADCA) or 6-aminopenicillanic acid (6-APA), respectively in the presence of a penicillin acylase, preferably an immobilized penicillin acylase. This enzymatic reaction may be carried according to any of the processes known in the art and which have been cited hereinbefore.

After the enzymatic coupling, the semi synthetic beta-lactam antibiotic can be recovered using known methods. For instance, the enzyme reactor may be discharged through the bottom sieve using upwards stirring. The resulting semi synthetic beta-lactam antibiotic suspension may then be filtered through a glass filter.

Due to the low amount of free side chain present after the enzymatic coupling reaction, crystallization of the final semi synthetic beta-lactam antibiotic may be carried out at high concentrations of the beta-lactam antibiotic which results in high yields.

### LEGEND TO THE FIGURES

FIG. 1 is the XRD spectrum of the hemi sulfuric acid salt of methyl (R)-2-amino-2-(cyclohexa-1,4-dien-1-yl)acetate, unambiguously showing the product to be in the crystalline state. X-axis: 2-theta value (deg). Y-axis: intensity (cps). The following distinct peaks can be discerned:

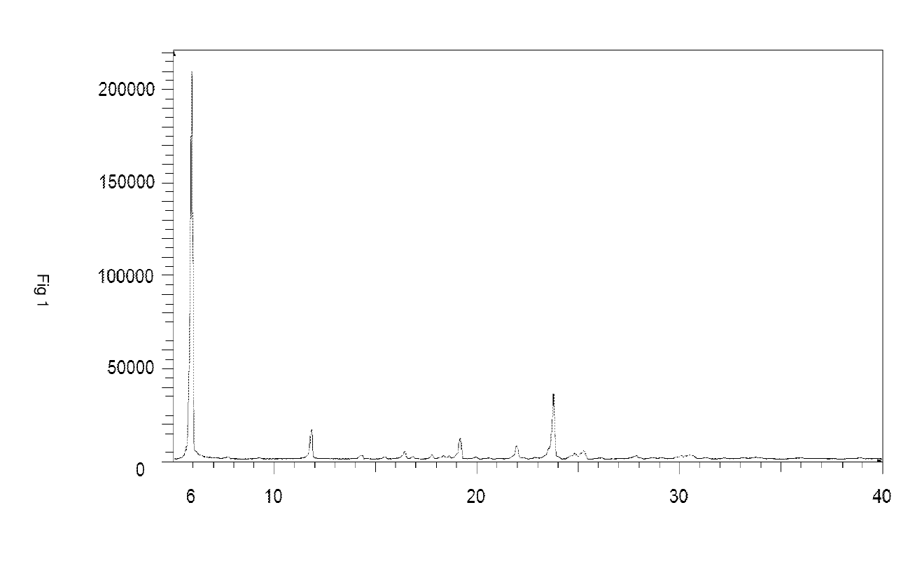

| | | | Intensity | |
|---|---|---|---|---|
| Peak no. | 2-Theta (deg) | d-Value (Angstrom) | (Count) | I/Io |
| 1 | 5.89 | 15.00 | 210356 | 100 |
| 2 | 11.80 | 7.49 | 17012 | 8 |
| 3 | 16.41 | 5.40 | 5463 | 3 |
| 4 | 19.16 | 4.63 | 12259 | 6 |
| 5 | 21.95 | 4.05 | 8293 | 4 |
| 6 | 23.77 | 3.74 | 36247 | 17 |
| 7 | 25.29 | 3.52 | 5477 | 3 |

### EXAMPLES

### General

### X-Ray Powder Diffraction Analysis

A sample was loaded onto a sample holder in a fume hood without grinding. The sample was analyzed on an X-ray powder diffractometer D2 Phaser from Bruker. It uses a LynxEye detector with 1°, opening angle, a 0.1 mm receiving slit and a nickel filter. The diffraction angle 2 theta ranges from 5°, to 40°, with step (in 20) ˜0.008°, and the count time 1 s/step. The sample rotates at 15 rpm during the measurement (for good statistics) io and the data were approximately background subtracted.

### HPLC Analysis

Equipment: high pressure liquid chromatograph Hewlett Packard model 1100
Column:Inertsil ODS 15 cm×4.6 mm, 5 μm
Mobile phase, solution pH=3.0: Phosphoric acid 85% (4.6 mL) was dissolved in water (1800 mL). The pH was adjusted to 3.0 using 5M NaOH and the final volume was adjusted to 2 L using water. A gradient with methanol was used (99.5% of the above solution with pH 3.0 and 5.0% of methanol).
Sample preparation and analysis: approximately 150 mg of sample was weighed and diluted to 100 mL with phosphate buffer pH=5.0 (buffer: KH₂PO₄ (5.44 g) was diluted to 2 L with water and the pH was adjusted to 5.0 using 1M KOH or H₃PO₄).

### Chromatographic Conditions:

Flow: 1 mL.min⁻¹
Injection volume: 25 μL
Wavelength: 220 nm
Temperature of column: room temperature, 25° C.
Retention times (approximately):

D-phenylglycine methyl ester (PGM): 10.0 min

Unknown 1: 10.9 min

Unknown 2: 11.9 min

methyl (R)-2-amino-2-(cyclohexa-1,4-dien-1-yl)acetate (DHPGM): 12.4 min

Unknown 3: 13.2 min

tetrahydro-D-phenylglycine methyl ester (THPGM): 17.5 min

### Preparation of methyl (R)-2-amino-2-(cyclohexa-1,4-dien-1-yl)acetate solution (DHPGM)

Under nitrogen atmosphere (R)-2-amino-2-(cyclohexa-1,4-dien-1-yl)acetic acid (D-dihydrophenylglycine, DHPG; about 1250-1500 kg) was suspended in methanol, at a ratio of 1 kg/1.1-2.0 L, while cooling down. The suspension was mixed with a mixture of methanol (about 0.7 L/kg DHPG) and 98% sulfuric acid (about 0.4 L/kg DHPG), while keeping temperature below 70° C. Subsequently, the mixture was heated to reflux and maintained for about 1-3 hours, then cooled concentrated under reduced pressure, until the temperature is less than 60° C. After cooling, methanol (about 0.3-1.5 L/kg DHPG) was added, and the mixture was heated to reflux and then distilled, as described above. This addition-reflux-distillation profile was repeated until a conversion of not less than 97% was reached. The mixture was then cooled. Ammonia (25%) was dosed until a pH of 1.7-3.4, maintaining temperature. Water was added (about 1 L/kg DHPG) and the solution was distilled under vacuum to remove methanol. Finally, the mixture comprising methyl (R)-2-amino-2-(cyclohexa-1,4-dien-1-yl)acetate (34.64%, pH=2) was cooled and stored for further use.

### Preparation of methyl (R)-2-amino-2-(cyclohexa-1,4-dien-1-yl)acetate free base (DHPGM free base; see also WO 2008/110527)

An aqueous solution of Na₂SO₄ (25%) was kept at 31° C. for later use. An aqueous solution of 2 M NaOH/5.3 M NaCl was pre-cooled in ice.

A vessel was pre-charged with a 5.3 M NaCl solution, sufficient to make contact with the agitator. The vessel was cooled in ice. The mixture (1002.5 g) comprising methyl (R)-2-amino-2-(cyclohexa-1,4-dien-1-yl)acetate obtained above was added drop wise into the vessel with co-current addition of 2 M NaOH/5.3 M NaCl, to maintain the pH at 9.2 while keeping the temperature <5° C. After all methyl (R)-2-amino-2-(cyclohexa-1,4-dien-1-yl)acetate was added, the mixture was heated to 25° C. and transferred to a separation funnel after which the aqueous phase was removed. The remaining organic phase was washed with 25% Na₂SO₄ twice (100 g and 96 g). The weight of the organic phase, being the free base of methyl (R)-2-amino-2-(cyclohexa-1,4-dien-1-yl)acetate (DHPGM free base) was 296.0 g.

### Example 1

### Preparation of the hemi sulfuric acid salt of methyl (R)-2-amino-2-(cyclohexa-1,4-dien-1-yl)acetate ((DHPGMH)₂SO₄)

A vessel with an agitator, cooled in an ice-bath, was pre-charged with DHPGM free base obtained as described in the General section (50 g). Pre-cooled H₂SO₄ (20% w/w) was added into the vessel with agitation until the pH was 4.2. After precipitates were observed, agitation was continued for another 30 min. The remainder of the DHPGM free base (246 g) was added to the vessel and co-currently also H₂SO₄ (20% w/w) so as to maintain the pH at 4.2 while keeping the temperature <5° C. Afterwards, agitation was maintained at 4° C. for 60 min. The crystals were isolated by filtration and washed with water. The wet cake (crop 1) thus obtained was dried under vacuum at 30° C. overnight to give 72 g of (DHPGMH)₂SO₄ with an assay of 89.7%. The mother liquor was stored at 4° C. overnight after which additional crystals formed. The crystals were isolated by filtration and washed with water. The wet cake (crop 2) thus obtained was dried under vacuum at 30° C. overnight to give 41 g of (DHPGMH)₂SO₄ with an assay of 90.6%.

Based on the HPLC peak areas, the ratios of the impurities to DHPGM (as 100%) in the different phases along the preparation of the (DHPGMH)₂SO₄ salt are as follows.

| | DHPGM | Unknown 1 | Unknown 2 | Unknown 3 | PGM | THPGM |
|---|---|---|---|---|---|---|
| | % based on HPLC peak area | | | | | |
| Starting Material | 100 | 0.7 | 0.1 | 0.3 | 8 | 3 |
| DHME free base | 100 | 0.7 | 0.1 | 0.3 | 8 | 3 |
| Aqueous phase | 100 | 0.8 | 0.1 | 0.4 | 8 | 3 |
| Mother liquor | 100 | 0.8 | 0.1 | 0.4 | 8 | 4 |
| Wet cake | 100 | 0.2 | 0.0 | 0.2 | 7 | 2 |
| Crystals crop1 | 100 | 0.2 | 0.1 | 0.2 | 9 | 2 |
| Crystals crop2 | 100 | 0.2 | 0.0 | 0.2 | 8 | 2 |

It was observed that, comparing with starting material, there were no extra impurities formed (DHPGM degradation) during the preparation of DHPGM free base, even with a large pH shift from 2 to 9. Unknown 1 was significantly reduced by the crystallization procedure, Unknown 3 and THPGM were slightly reduced.

In a separate analogous experiment, 16 g of (DHPGMH)₂SO₄ with an assay of 93% was prepared.

### Example 2

### Preparation of cephradine using (DHPGMH)₂SO₄ vs DHPGM in solution

7-Aminodeacetoxycephalosporanic acid (7-ADCA, 50.0 g) was suspended in water (153 g) and the temperature was controlled at 20° C. The mixture was stirred for 5 min while maintaining the pH at 6.9 by the addition of an aqueous solution of ammonia (25%). Immobilized enzyme (comprising mutant 1 as described in U.S. Pat. No. 8,541,199; 55 g) was added together with water (60.5 g). Next, solid (DHPGMH)2504 (54.9 g) was dosed is at a constant rate in 150-180 min. whilst the pH was maintained at 6.9 by the addition of an aqueous solution of ammonia (25%) or with an aqueous solution of sulfuric acid (30%) once all (DHPGMH)2504 was added. After 210-240 min., the conversion as >93.5%, the suspension was cooled to 5° C. in 20 min. while maintaining the pH at 6.9. Subsequently the pH was lowered to 6.0 with sulfuric acid (30%). During the course of the reaction samples were taken and analyzed by HPLC with the results as outlined in Table 1.

TABLE 1
Formation of cephradine from 7-ADCA using solid (DHPGMH)₂SO₄

| Time | DHPGM | 7-ADCA | DHPG | Cephradine | Cephalexin | PG | Conversion | | |
|---|---|---|---|---|---|---|---|---|---|
| (min) | (%) | (%) | (%) | (%) | (%) | (%) | (%) | Ratio | S/H |
| 90 | 0.30 | 3.16 | 0.13 | 7.98 | 0.21 | 0.02 | 69.9 | 0.686 | 26.9 |
| 120 | 0.31 | 2.44 | 0.18 | 10.32 | 0.18 | 0.03 | 76.5 | 0.803 | 25.1 |
| 150 | 0.41 | 1.86 | 0.22 | 13.28 | 0.24 | 0.04 | 82.0 | 0.904 | 26.5 |
| 180 | 0.48 | 0.81 | 0.27 | 16.14 | 0.31 | 0.04 | 92.1 | 1.022 | 26.2 |
| 210 | 0.11 | 0.47 | 0.34 | 15.97 | 0.27 | 0.06 | 95.4 | 1.022 | 20.6 |
| 270 | 0.12 | 0.63 | 0.41 | 17.40 | 0.31 | 0.07 | 93.9 | 1.017 | 18.6 |
| (5° C.) | | | | | | | | | |
| pH 6.0 | 0.11 | 0.61 | 0.36 | 15.10 | 0.23 | 0.08 | 94.1 | 1.015 | 18.4 |

Components are given in weight %
Conversion: 100*moles cephradine/(moles cephradine + 7-ADCA)
Ratio: (moles cephradine + DHPGM + DHPG)/(moles cephradine + 7-ADCA)
S/H: Synthesis/Hydrolysis ratio, or moles cephradine/moles DHPG

For comparative reasons the above cephradine protocol was repeated however using DHPGM solution (34.64%, pH=2, as obtained in the above General section) instead of solid (PGMH)₂SO₄, and less water as outlined in WO 2005/003367 to compensate for the water present in the DHPGM solution. During the course of the reaction samples were taken and analyzed by HPLC with the results as outlined in Table 2.

TABLE 2
Formation of cephradine from 7-ADCA using DHPGM in solution

| Time | DHPGM | 7-ADCA | DHPG | Cephradine | Cephalexin | PG | Conversion | | |
|---|---|---|---|---|---|---|---|---|---|
| (min) | (%) | (%) | (%) | (%) | (%) | (%) | (%) | Ratio | S/H |
| 90 | 0.78 | 6.68 | 0.13 | 9.19 | 0.17 | 0.03 | 38.8 | 0.561 | 31.0 |
| 120 | 0.33 | 5.09 | 0.16 | 11.19 | 0.32 | 0.02 | 53.1 | 0.637 | 30.7 |
| 150 | 0.80 | 3.32 | 0.22 | 13.10 | 0.40 | 0.03 | 69.2 | 0.832 | 26.1 |
| 181 | 0.47 | 1.24 | 0.29 | 15.29 | 0.44 | 0.03 | 88.5 | 0.983 | 23.1 |
| 210 | 0.16 | 0.76 | 0.33 | 16.22 | 0.44 | 0.04 | 92.9 | 0.997 | 21.5 |
| 240 | 0.10 | 0.62 | 0.32 | 16.55 | 0.45 | 0.03 | 94.2 | 1.000 | 22.7 |
| 300 | 0.06 | 0.56 | 0.31 | 16.24 | 0.48 | 0.04 | 94.7 | 1.001 | 23.0 |
| (5° C.) | | | | | | | | | |
| pH 6.0 | 0.07 | 0.56 | 0.40 | 16.11 | 0.44 | 0.05 | 94.7 | 1.015 | 17.7 |

Legend: As in Table 1

Inspection of Tables 1 and 2 revealed that the use of solid (DHPGMH)₂SO₄ resulted in better results over the use of DHPGM in solution, in terms of speed of conversion, formation of (unwanted) cephalexin and overall S/H ratio end of reaction.

### Prophetic Example 3

### Preparation of cefradoxine using (DHPGMH)₂SO₄ vs DHPGM in solution

7-Amino-3-methoxy-3-cephem-4-carboxylic acid (7-AMOCA, 234 mmol) is suspended in water (153 g) and the temperature is controlled at 20° C. The mixture is stirred for 5 min while maintaining the pH at 6.7 by the addition of an aqueous solution of ammonia (25%). Immobilized enzyme (comprising mutant 1 as described in U.S. Pat. No. 8,541,199; 55 g) is added together with water (60.5 g). Next, solid (DHPGMH)₂SO₄ (54.9 g) is dosed at a constant rate in 150-180 min. whilst the pH was maintained at 6.9 by the addition of an aqueous solution of ammonia (25%) or with an aqueous solution of sulfuric acid (30%) once all (DHPGMH)₂SO₄ is added. After the conversion is >93.5%, the suspension is cooled to 5° C. in 20 min. while maintaining the pH at 6.9. Subsequently the pH is lowered to 6.0 with sulfuric acid (30%). During the course of the reaction samples are taken and analyzed by HPLC.

For comparative reasons the above cephradine protocol was repeated however using DHPGM solution (34.64%, pH=2, as obtained in the above General section) instead of solid (PGMH)₂SO₄, and less water as outlined in WO 2005/003367 to compensate for the water present in the DHPGM solution.

### Prophetic Example 4

### Preparation of epicillin using (DHPGMH)₂SO₄ vs DHPGM in solution

6-Aminopenicillanic acid (6-APA, 234 mmol) is suspended in water (153 g) and the temperature is controlled at 20° C. The mixture is stirred for 5 min while maintaining the pH at 6.7 by the addition of an aqueous solution of ammonia (25%). Immobilized enzyme (comprising mutant 1 as described in U.S. Pat. No. 8,541,199; 55 g) is added together with water (60.5 g). Next, solid (DHPGMH)₂SO₄ (54.9 g) is dosed at a constant rate in 150-180 min. whilst the pH was maintained at 6.9 by the addition of an aqueous solution of ammonia (25%) or with an aqueous solution of sulfuric acid (30%) once all (DHPGMH)₂SO₄ is added. After the conversion is >93.5%, the suspension is cooled to 5° C. in 20 min. while maintaining the pH at 6.9. Subsequently the pH is lowered to 6.0 with sulfuric acid (30%). During the course of the reaction samples are taken and analyzed by HPLC.

For comparative reasons the above cephradine protocol was repeated however using DHPGM solution (34.64%, pH=2, as obtained in the above General section) instead of solid (PGMH)₂SO₄, and less water as outlined in WO 2005/003367 to compensate for the water present in the DHPGM solution.

1. The hemi sulfuric acid salt of methyl (R)-2-amino-2-(cyclohexa-1,4-dien-1-yl)acetate.

2. The hemi sulfuric acid salt according to claim 1 having an XRD powder diffraction pattern comprising peaks at 5.9±0.2 degrees 2-theta, 11.8±0.2 degrees 2-theta, 19.2±0.2 degrees 2-theta and 23.8±0.2 degrees 2-theta.

3. The hemi sulfuric acid salt according to claim 2 further comprising peaks at 16.4±0.2 degrees 2-theta, 22.0±0.2 degrees 2-theta and 25.3±0.2 degrees 2-theta.

4. An aqueous solution comprising the hemi sulfuric acid salt according to claim 1.

5. Method for the preparation of the hemi sulfuric acid salt of methyl (R)-2-amino-2-(cyclohexa-1,4-dien-1-yl)acetate comprising the steps of:
(a) contacting methyl (R)-2-amino-2-(cyclohexa-1,4-dien-1-yl)acetate free base with sulfuric acid;
(b) isolating the hemi sulfuric acid salt of methyl (R)-2-amino-2-(cyclohexa-1,4-dien-1-yl)acetate from the mixture obtained in step (a),
characterized in that in step (a) the molar amount of sulfuric acid is from 0.4 to 0.6 relative to the molar amount of methyl (R)-2-amino-2-(cyclohexa-1,4-dien-1-yl)acetate.

6. Method according to claim 5 wherein said isolating in step (b) is achieved by centrifugation, filtration or sedimentation of crystalline hemi sulfuric acid salt of methyl (R)-2-amino-2-(cyclohexa-1,4-dien-1-yl)acetate.

7. Method according to claim 5 wherein step (b) is preceded by lowering of the temperature to −5° C. to 15° C.

8. A method of preparing epicillin, cephradine or cefroxadine comprising contacting said hemi sulfuric acid salt of methyl (R)-2-amino-2-(cyclohexa-1,4-dien-1-yl)acetate with 6-aminopenicillanic acid, 7-aminodeacetoxycephalosporanic acid or 7-amino-3-methoxy-3-cephem-4-carboxylic acid, respectively, in the presence of a penicillin acylase.
